(19) United States
(12) Patent Application Publication
(10) Pub. No.: US 2009/0149608 A1
(43) Pub. Date: Jun. 11, 2009

(54) Polycarbamides, polycarbamates, and polycarbamide-formaldehyde and polycarbamate-formaldehyde condensation resins

(76) Inventor: Moon Kim, Starkville, MS (US)

Correspondence Address: Larry A. Schemmel, Office of the Attorney General, P.O. Box 1850, Jackson, MS, 39215-1850, US

(21) Appl. No.: 12/001,056

(22) Filed: Dec. 7, 2007

(51) Int. Cl. C08L 67/00; C07C 271/02; C08G 69/02

(52) U.S. Cl. 525/418; 560/158; 528/310

(57) ABSTRACT

The present invention provides manufacturing of and the use of novel polycarbamates and polycarbamides and their condensation reaction products formed by reacting with formaldehyde as wood composite binder resins and in other applications. These resins have thermosetting capabilities and usefulness as binders for wood and other materials with superior resin properties of low cost, colorlessness, exceptionally good binding, and fast curing characteristics, as well as very low formaldehyde emissions. The newly-designed and synthesized novel starting materials for the thermosetting resins of the present invention are diethylene tricarbamide and various polycarbamates derived from corresponding polyols.

### STATEMENT OF GOVERNMENT SUPPORT

This invention was made with government support under grant 2002-34158-11926 awarded by the U.S. Department of Agriculture WUR. The government may have certain rights in the invention.

### FIELD OF THE INVENTION

The present invention relates to the field of wood composite binder resins and other areas of application. In particular, the present invention relates to manufacturing and using novel polycarbamates and polycarbamides and their condensation reaction products formed by reacting with formaldehyde, each of which have thermosetting capabilities and usefulness as binders for wood and other materials as well as in other applications.

### BACKGROUND OF THE INVENTION

Urea-formaldehyde (UF) resin adhesives are commonly used to produce wood composite products such as particleboard, medium-density fiberboard, and hardwood plywood panels. These UF resins are considered good binders in these applications due to high physical strength properties, faster curing times, and high cost-efficiency. Two major drawbacks to UF resin-based systems, however, are the limited strength durability of the resulting composite products as well as the emission of formaldehyde. Formaldehyde emissions are of particular concern when using UF resin-bonded boards for interior purposes such as sub-flooring, shelving, cabinets, and furniture. Air concentrations of formaldehyde above 0.1 parts per million (ppm) are associated with acute health effects, including watery eyes, burning sensations in the eyes, nose and throat, nausea, coughing, chest tightness, wheezing, skin rashes, headaches, fatigue, asthma, and other irritating effects. Formaldehyde has been shown to be cancer-causing in laboratory animals, although there is limited evidence of cancer-causing effects in humans. Nevertheless, it is classified as a “probable human carcinogen” by the United States Environmental Protection Agency (EPA) and the National Institute for Occupational Safety and Health.

Both the formaldehyde emission problem and the durability issues of UF resin-bonded wood products are linked to the underlying chemistry of the UF resin system. During synthesis of resin, hydroxymethyl groups are formed from the reaction of formaldehyde (F) and urea (U) as functional groups needed for the subsequent polymerization and curing processes. However, the reverse reaction of hydroxymethyl group formation also occurs during synthesis and subsequent curing processes to generate back some free formaldehyde, which is later emitted into the environment. The extent of the reverse reaction is generally proportional to the F/U mole ratio used in resin synthesis and is relatively small in comparison to the forward reaction, but still persists to the current low F/U molar ratio for resins of about 1.15 (Myers, G. E. Holzforschung 44:117-126 (1990); Forest Products Journal 34:35-41 (1984). This is the underlying mechanism for the formaldehyde emission phenomena of UF resin-bonded wood composite boards. This low F/U mole ratio of resin needed for lower emission, on the other hand, translates into a functionality value of about 2.3 formaldehyde molecules per urea molecule in current UF resins. Polymer molecular theory on the formaldehyde-based thermosetting resins indicates that the base monomer (for example, urea) needs to have a functionality of at least 3.0 or higher to make the resin polymers grow to a three-dimensional, fully cross-linked state (Flory, P. J. Polymer Chemistry, Cornell University Press, Ithaca, N.Y. (1953) p. 79). Since the urea functionality in current UF resins is significantly lower than the theoretical value, a full cross-linking does not occur and the cured resin binders will result in limited strength durability of boards. The formaldehyde emission problem still persists at the current F/U mole ratio values of resin. Currently, UF resin formulation (mostly lowering of F/U mole ratio) and scavenger parameters have been pushed to limits for reduction of formaldehyde emission from boards, but significant further formaldehyde emission reductions are desired. From the above theoretical consideration, such a reduction in formaldehyde emission level for UF resin-bonded boards seems to require a significant redesigning of the starting molecule toward materials having higher functionality than urea. There accordingly remains a need in the art for interior-grade wood composite binder resins for improved starting materials as well as their formaldehyde condensation products that give superior resin properties of low cost, colorlessness, exceptionally good binding, and fast curing characteristics, as well as very low formaldehyde emissions. The present invention provides such advantages.

### SUMMARY OF THE INVENTION

It is an object of the present invention to provide manufacturing of and the use of novel polycarbamates and polycarbamides and their condensation reaction products formed by reacting with formaldehyde as wood composite binder resins and in other applications.

The newly-designed and synthesized novel starting materials for the thermosetting resins of the present invention are diethylene tricarbamide and various polycarbamates derived from corresponding polyols: glycerol tricarbamate, 1,1,1-trihydroxymethyl ethane tricarbamate, 1,1,1-trihydroxymethyl propane tricarbamate, meso-erythritol tetracarbamate, pentaerythritol tetracarbamate, xylitol pentacarbamate, D-sorbitol hexacarbamate, and D-mannitol hexacarbamate. Ethylene dicarbamide and ethylene glycol dicarbamate, known compounds, were also found useful as resin synthesis starting materials. These polycarbamides and polycarbamates are reacted with formaldehyde at elevated temperatures in weak alkaline and then optionally in weak acidic pH, or only in weak acidic pH, to result in thermosetting resins useful in many applications, including binders for wood composite boards such as particleboard, medium density fiberboard, hardwood plywood, and others with improved board strengths while having very low potentials of formaldehyde emission. Another advantage of the resin materials of the present invention is the inter-miscibility of variously synthesized, different carbamide-formaldehyde and carbamate-formaldehyde resins and also with urea or melamine in any proportions to take advantages of lower cost or lower formaldehyde emission.

The thermosetting resin materials of the present invention with an acid-generating latent catalyst and with or without other filler additive materials are applied on substrates and cured at elevated temperatures of about 120° C.-300° C. until hardened. The cured resin materials show good stability at the curing temperatures and also good durability and strength after cooling to room temperature to be useful as adhesives, impregnating matrix binders, treatment chemicals, and other areas where high strength/weight ratios are needed. The handling and curing properties of resins of the present invention are especially suited to industrial thermosetting processes including manufacturing wood composite boards such as particleboard, medium density fiberboard, hardwood and softwood plywood, oriented strand board, strawboard, and the like and treatments of paper, cotton textiles, leather, cardboard, felt, sand mold, and the like. The resins of this disclosure can be useful as binders for non-woven materials such as paper, cotton, leather, cardboard, and other felt products to improve the wet and dry strengths and also can be useful as binders for sand molds in the metal casting industry. The polycarbamides and polycarbamates and polycarbamide- and polycarbamate-formaldehyde condensation products of the present invention are quite unique and novel and likely useful in many industrial processes. The diethylene tricarbamide and all polycarbamates of the present invention are for the first time synthesized and found to be useful as starting materials of thermosetting resins and may also be used in areas other than manufacturing of formaldehyde condensation products. It is to be understood that changes and variations may be made without departing from the spirit and scope of the invention as defined in the appended claims.

With the foregoing and other objects, features, and advantages of the present invention that will become apparent hereinafter, the nature of the invention may be more clearly understood by reference to the following detailed description of the preferred embodiments of the invention and to the appended claims.

### BRIEF DESCRIPTION OF THE DRAWINGS

These drawings accompany the detailed description of the invention and are intended to illustrate further the invention and its advantages:

FIG. 1 is a graphical illustration of a typical ¹³C NMR spectrum for the GCAF resin of Example 1b.

FIG. 2 is a graphical illustration of the DMA curing results of the GCAF resin of Example 1b.

### DETAILED DESCRIPTION OF THE INVENTION

The present invention provides for the manufacturing of and the use of novel polycarbamates and polycarbamides and their condensation reaction products formed by reacting with formaldehyde as wood composite binder resins and in other applications. Additional objectives and advantages of the present invention are to provide products with exceptional thermosetting capabilities and usefulness as binders for wood and other materials. It will be understood by those skilled in the art that the present invention is not limited in its application to the details of the arrangements described herein since it is capable of other embodiments and modifications. Moreover, the terminology used herein is for the purpose of such description and not of limitation.

### Synthesis Methods of Ethylene Dicarbamide and Diethylene Tricarbamide

Ethylenediamine (NH₂—CH₂CH₂—NH₂) and diethylenetriamine (NH₂—CH₂CH₂—NH—CH₂CH₂—NH₂)), both known polyamine compounds, were reacted either with urea (Method 1) or sodium cyanate (Method 2) to obtain, respectively; ethylene dicarbamide (known compound) and diethylene tricarbamide in good yields. The procedures were adapted from methods known for simple monoamines. Both synthesized polycarbamides were found to be very stable under ordinary conditions as well as in heating or mild acid or base treatments.

Method 1—In this procedure, the polyamine is reacted with a slight molar excess of urea in the presence of water as a solvent to obtain the corresponding polycarbamides by splitting off ammonia:

R—(NH₂)ₙ+nNH₂CONH₂→R—(NHCONH₂)ₙ+NH₃

This method is well known for simple amines (Organic Synthesis 3, 95 (1923)). In this disclosure, diethylenetriamine is reacted with urea as follows: In a 500 mL three-neck flask equipped with a stirrer, condenser, and thermometer, 90.0 grams of diethylenetriamine (0.87 mole) were dissolved in 2.5 L of water and then 210.0 grams of urea (3.5 moles) were added to the mixture. Then, the stirred reaction mixture was heated to 100-104° C. over a period of 30 min and allowed to react for an hour, followed by allowing a slow distillation of water containing ammonia for three hours. Finally, the distillation was continued under water-vacuum for 30 min and then the remaining solution was poured off on to a pan and allowed to cool to room temperature. The separated colorless precipitates were collected as crude diethylene tricarbamide, which can be purified by dissolving in water at elevated temperatures followed by cooling and filtration and drying. The analysis results are recorded in Table 1.

Method 2—In this procedure, the polyamine is reacted with sodium cyanate (NaOCN) in the presence of an acid (HX) and water as a solvent to obtain the corresponding polycarbamide by splitting off sodium salt of the acid:

R—(NH₂)ₙ+nNaOCN+HX→R—(NHCONH₂)ₙ+NaX

This method is well known for monoamines (Vogel, A. I. Practical Organic Chemistry 3^{rd} Ed., Longman, London (1972), p. 644). In this disclosure, ethylenediamine is reacted with sodium cyanate as follows: In a 500 mL three-neck flask equipped with a stirrer, condenser, and thermometer, 60.1 grams of ethylenediamine (1.0 mole) were dissolved in 300 mL of water and then 98.0 grams of sulfuric acid (1.0 mole) were added to the mixture with external cooling to about 50° C. to 90° C., preferably to about 65° C. Then, to the stirred, warm reaction mixture, 137 grams of sodium cyanate (2.1 moles) were added over a period of 30 min, followed by reacting an additional hour at the same temperature. Finally, the reaction mixture were cooled to room temperature and the separated colorless precipitates collected as crude ethylene dicarbamide, which was purified by dissolving in water at elevated temperatures followed by cooling and filtration and drying with a melting point of 190-195° C. and correct analyzed by ¹³C NMR and infrared spectra and carbon, hydrogen, and nitrogen elemental analysis. The acid (HX) can be any inorganic or organic acid such as sulfuric, phosphoric, nitric, hydrochloric, formic, acetic, and oxalic acid.

### Synthesis Methods of Various Polycarbamates

The polycarbamates synthesized as starting materials of thermosetting resins of the present invention were all derived from polyols as follows: glycerol tricarbamate, 1,1,1-trihydroxymethyl ethane tricarbamate, 1,1,1-trihydroxymethyl propane tricarbamate, meso-erythritol tetracarbamate, pentaerythritol tetracarbamate, xylitol pentacarbamate, D-sorbitol hexacarbamate, and D-mannitol hexacarbamate. These polycarbamates were first synthesized in our laboratory using three different methods adapted from the procedures known for monoalcohols (Method 3, Method 4, and Method 5, herein) in good yields and found to be very stable under ordinary conditions as well as in heating or mild acid or base treatments. Some of the starting polyols are derived from simple sugars through a simple hydrogenation process and simple sugars are the most plentiful, renewable materials obtained from hydrolysis of polysaccharides such as starch, cellulose, and other plant and animal-derived raw materials.

Method 3—In this procedure, the polyol is reacted with phenyl chloroformate in the presence of a tertiary amine such as pyridine to capture generated hydrogen chloride in a solvent such as tetrahydrofuran (THF) to obtain the polyol-phenyl carbonate intermediate and then the intermediate is reacted with ammonia (or concentrated ammonium hydroxide solution) to obtain the polycarbamate by splitting off phenol:

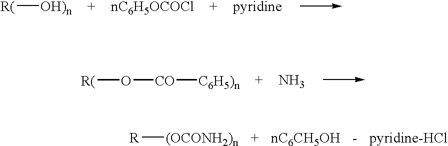

This method is well known for monoalcohols (Adams, P. and F. A. Baron, Chemical Reviews 65, 567-602 (1965)). In this disclosure, glycerol tricarbamate was synthesized as follows: In a 500 mL three-neck flask equipped with a stirrer, condenser, and thermometer, 14.8 grams of glycerol (0.161 mole) were dissolved in a mixture of 57.0 grams of pyridine (0.73 mole) and 150 mL of THF. Then, 76.0 grams of phenyl chloroformate (0.484 mole) were added to the stirred reaction mixture over a period of one hour through a dropping funnel while keeping the reaction temperature below about 60° C. The reaction mixture was allowed to stir for an hour and then cooled to room temperature over a period of an hour and the formed pyridine hydrochloride crystals were filtered off. The filtrate containing the “carbonate intermediate” was then charged into a three-neck flask equipped with a stirrer and thermometer and ammonia gas (or ammonium hydroxide) was introduced into it over a period of one hour to saturation. The reaction mixture was allowed to stir for two more hours and then the solid precipitates of glycerol tricarbamate were collected by filtration. The crude product was purified by suspension in water followed by re-filtration and drying.

Method 4—This procedure was adapted from the known method of synthesizing various monocarbamates (Kosovsky, P. Tetrahedron Lett, 27: 5521 (1986)) from mono-alcohols by using commercially available trichloracetyl isocyanate:

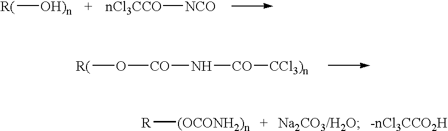

In this disclosure, the procedure for glycerol tricarbamate is as follows: In a 100 mL three-neck flask equipped with a stirrer and thermometer, 1.46 grams of glycerol (0.016 mole) were dissolved in acetone and the reactor is cooled in an ice/water bath and then 10.0 grams of trichloroacetyl isocyanate (0.053 mole) were added in drops over a period of 15 minutes while stirring. The reaction mixture was allowed to warm up to room temperature over a period of 30 min and stirred for one hour at room temperature. The reaction mixture was then taken in a flask and the acetone was evaporated on a rotary evaporator and the residue was taken in a mixture of 40 mL methanol and 10 mL water and 0.50 gram of sodium carbonate was added to it and stirred at 50° C. in a water bath. The insoluble materials were collected by filtration and the crude product was purified by re-crystallization from hot water.

Method 5—In this procedure for synthesis of polycarbamates, the polyol is reacted with phosgene in the presence of a small amount of tertiary amine such as pyridine to catalyze the reaction with or without a solvent such as tetrahydrofuran (THF) to obtain the chloroformate of polyol as intermediate, which is then reacted with excess ammonia (or concentrated ammonium hydroxide solution) to obtain the polycarbamate by splitting off ammonium chloride:

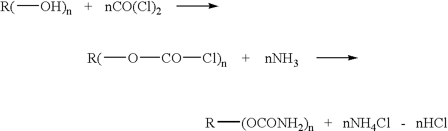

This procedure is well known for monoalcohols (Marzner, M., R. P. Kurkjy and R. J. Cotter, Chemical Review 64, 645-687 (1964); Sonntag, N. O., Chemical Review 52, 258-294 (1953)). The first step of synthesizing chloroformates is also known. In this disclosure, glycerol tricarbamate was synthesized as follows: In a 50 mL three-neck flask equipped with a stirrer, condenser, thermometer and an inlet of dry nitrogen gas, 50 ml tetrahydrofuran was charged and cooled to −20° C. and then 5.0 grams of phosgene (0.0505 mole) were condensed into the reactor. Then, while stirring, 0.040 gram of pyridine (0.0005 mole) was added and kept stirring so that the formed pyridine hydrochloride particles were well dispersed in the solution. Then, while a small stream of dry nitrogen was being continuously introduced, 1.48 grams of glycerol (0.016 mole) was added in small amounts over a period of two hours while the formed hydrogen chloride was swept off through the condenser. The reaction temperature was maintained in −20° C.˜15° C. in this period. Then, when the hydrogen chloride evolution was completed, the whole solution of glyceryl trichloroformate was treated with saturated ammonium hydroxide solution and the solid precipitates of glycerol tricarbamate were collected by filtration. The crude product was purified by re-crystallization from hot water. This method and process is also applicable to preparing meso-erythritol tetracarbamate, D-xylitol pentacarbamate, D-sorbitol hexacarbamate, D-mannitol hexacarbamate, 1,1,1-trihydroxymethylethane tricarbamate, 1,1,1-trihydroxymethylpropane tricarbamate, and pentaerythritol tetracarbamate.

All newly synthesized polycarbamides and polycarbamates of the present invention were found to be stable at room temperature and in heating or mild acid or base treatments in water or common organic solvent. The synthesis yields obtained, chemical structures identified by ¹³C NMR, meting points, major infrared spectroscopy peaks, and elemental analysis results for the synthesized polycarbamides and polycarbamates are shown in Table 1 as proof of syntheses and the chemical structural identities.

TABLE 1

| | | Synthesized | | | | Carbon, nitrogen, | | |
|---|---|---|---|---|---|---|---|---|
| Starting polyols or | | carbamides and | ¹³C NMR | Melting | Major ir | Hydrogen analysis | | |
| polyamine | Yield | polycarbamates | chemical shift | Points | Peaks | (%): Theory/Observed | | |
| Structures/Names | (%) | Structures/Names | (ppm) | ° C. | cm⁻¹ | C | H | N |
| NH₂CH₂CH₂NH— | | NH₂CONHCH₂— | 159.26:38.53 | 217-219 | 3420; | 36.20/ | 6.95/ | 36.19/ |
| CH₂CH₂NH₂ | | CH₂N(CONH₂)CH₂— | 47.44:158.70:47.44 | | 3370; 3220 | 35.47 | 6.98 | 35.62 |
| Diethylenetriamine | | CH₂NHCONH₂ | 38.53:159.26 | | 2950; | | | |
| | | Diethylene tricarbamide | | | 1660; 1560 | | | |
| 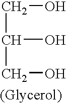 | 76% | 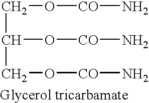 | 62.30:156.28 69.62:155.88 62.30:156.28 | 167-171 | 3420 3415 2985 1710 1660; 1560 | 32.58/ 32.57 | 5.01/ 5.49 | 19.00/ 18.92 |
| 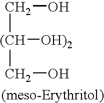 | 75% | 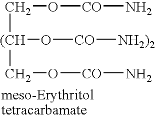 | 62.03:156.47 (70.39:155.89)₂ 62.03:156.47 | 275-276 | 3420-3415 2985; 1710 1675; 1645 1610 | 32.66/ 32.69 | 4.80/ 4.82 | 19.04/ 18.95 |
| 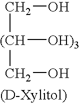 | 70% | 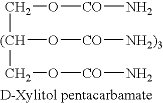 | 62.77:156.43 (69.71:156.04)₃ 62.77:156.43 | 257-259 | 3420-3415 2985; 1710 1675; 1610 | 32.70/ 32.75 | 4.67/ 4.80 | 19.07/ 18.56 |
| 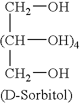 | 70% | 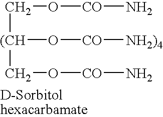 | 62.59:156.43 69.52:155.79 69.88:155.66 69.88:155.66 69.88:155.59 61.92:156.35 | 277-280 | 3420-3415 2985; 1710 1675; 1640 1610 | 32.73/ 32.56 | 4.58/ 4.76 | 19.09/ 18.40 |
| 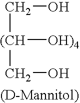 | 76% | 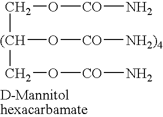 | 61.97:156.47 70.24:155.82 70.13:155.61 70.13:155.61 70.24:155.82 61.97:156.47 | 290-305 | 3420-3415 2985; 1710 1670; 1640 1610 | 32.71/ 32.83 | 4.58/ 4.89 | 19.09/ 17.71 |
| CH₃—C—(CH₂—OH)₃ | 78% | CH₃C— | 33.14:38.72 | 175-179 | 3420-3415 | 38.55/ | 6.07/ | 16.86/ |
| {1,1,1- | | (CH₂—O—CO—NH₂)₃ | (65.21:156.70)₃ | | 2985; 1715 | 38.54 | 6.14 | 16.88 |
| Tri(hydroxymethyl)- | | 1,1,1-Tri(hydroxymethyl)- | | | 1690; 1610 | | | |
| ethane} | | ethane tricarbamate | | | | | | |
| CH₃CH₂—C—(CH₂OH)₃ | 78% | CH₃CH₂—C— | 7.47:22.50:40.80: | 162-164 | 3420-3415 | 41.06/ | 6.51/ | 15.96/ |
| {1,1,1- | | (CH₂—O—CO—NH₂)₃ | (63.4:156.71)₃ | | 2985; 1710 | 41.07 | 6.58 | 15.98 |
| Tri(hydroxymethyl)- | | 1,1,1-Tri(hydroxymethyl)- | | | 1685; 1595 | | | |
| propane} | | propane tricarbamate | | | | | | |
| C—(CH₂—OH)₄ | 82% | C— | 42.24: | 270-274 | 3420-3415 | 35.01/ | 5.23/ | 18.18/ |
| (Pentaerythritol) | | (CH₂—O—CO—NH₂)₄ | ((62.09:156.45)₄ | | 2985; 1710 | 34.92 | 5.35 | 17.88 |
| | | Pentaerythritol | | | 1675; 1600 | | | |
| | | tetracarbamate | | | | | | |

In conclusion, the synthesized polycarbamides and the polycarbamates of the present invention shown in Table 1 were new compounds synthesized for the first time and the chemical structures are fully characterized through the synthetic procedures and various analytical results. All compounds in Table 1 are novel in that the molecules are composed of organic carbon-chain backbones and have three or more amide or carbamate functional groups in a molecule for reaction with formaldehyde. Comparing to urea's two carbamide functional groups in a molecule, the greater number of functionality of the starting materials of the present invention have manifested the novelty in reaction with formaldehyde and the reaction products' resin properties and cured polymers' usefulness as adhesives and other applications as demonstrated in the Examples below.

Synthesis Methods of Polycarbamide-Formaldehyde (CIF) and Polycarbamate-Formaldehyde (CAF) Resins with No Poly-Condensation Step

Synthesis of the thermosetting resins of the present invention was accomplished by reacting all synthesized polycarbamates and diethylene tricarbamide in Table 1 as well as ethylene glycol dicarbamate and ethylene dicarbamide with formaldehyde, resulting in useful thermosetting resins. In the typical resin synthesis procedure, an appropriate amount of 50% formaldehyde solution is charged into a stirred reactor equipped with a thermometer and condenser along with water to keep the resin solids level to the common 50%-60% range. The pH of the formaldehyde solution is then adjusted to 5.5-9.0 and heating is applied to heat the reactor to about 80° C.-90° C. Then, the solid polycarbamate or polycarbamide is added in small portions over a period of 20-30 min and heating is continued to maintain the reaction mixture at 90° C.-106° C. for 15 min or longer depending on the polycarbamate or polycarbamide until the reaction mixture becomes clear. The condensation reaction is continued for 0.5-2 hours at the same temperature with the pH of the reaction mixture being maintained at 7-10 for completion of the condensation reaction. The concentration of formaldehyde and polycarbamate or polycarbamide in the initial reaction mixture should be between 10% and 90%. The mole ratio of formaldehyde to polycarbamate or polycarbamide in the reaction should be between 0.1 and 1.5 moles of formaldehyde per each carbamate or carbamide group with the preferred ratio being 0.2 to 1.2 moles. Furthermore, any combination of two or more polycarbamates or polycarbamide can be used instead of one. The formaldehyde can be in any form, commonly 37% to 60% aqueous solutions or solid paraformaldehyde, as long as the overall levels of reactants are maintained by using an appropriate amount of water. However, the 50% formaldehyde solution is commonly used in the thermosetting resin manufacturing industry. The temperature of the reaction may be varied from 30° C. to the boiling point of the reaction mixture, which may go as high as 105° C. under normal atmospheric pressure. After completion of the reaction described above, the product is cooled to room temperature and can be used directly with or without some additional acidic catalysts. When the reaction products need to be stored or transported, the pH must be adjusted to 5.0-9.0 by adding a suitable alkaline material such as ammonia, sodium or potassium hydroxide or carbonate. Analyses of the polycarbamide-formaldehyde (CIF) and carbamate-formaldehyde (CAF) condensation reaction products using ¹³C NMR spectroscopy indicated the almost quantitative formation of hydroxymethyl groups bonded to the carbamide or carbamate groups in accord with the chemistry known for monocarbamates and for urea in literature (Marvel, C. S., J. R. Elliot, F. E. Boettner, and H. Yasuka, J. Amer. Chem. Soc. 68, 1681 (1946); Kim, M. G. J. Polymer Science, Part A: Polymer Chemistry, 37: 995-1007 (1999)).

### Syntheses of Oligomeric Carbamide-Formaldehyde (CIF) and Carbamate-Formaldehyde (CAF) Resins by Further Condensation in Acidic pH

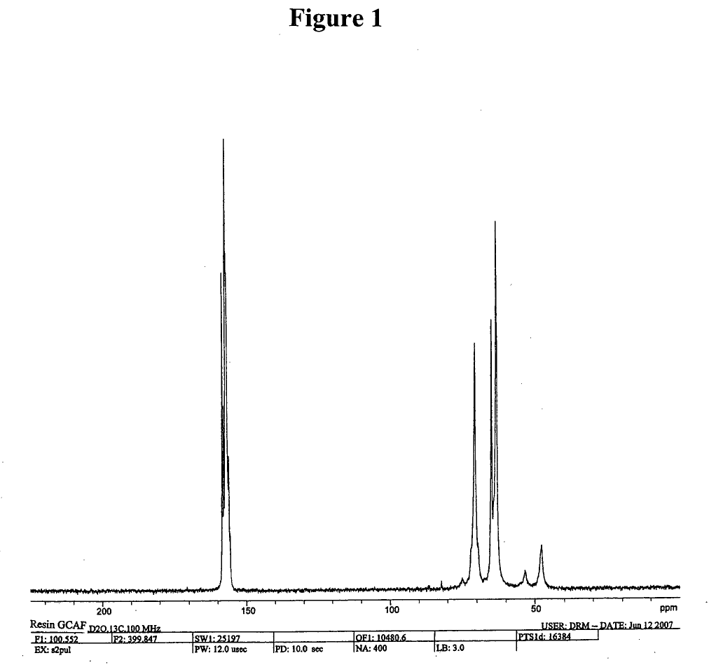

For preparation of higher molecular weight oligomeric polycarbamide-formaldehyde and polycarbamate-formaldehyde resin products, the condensation reaction product described above is acidified by adding a dilute solution of a strong acid such as sulfuric acid or hydrochloric acid (˜8%) to pH 0.5-6.9 and then reacted at about 30° C.-105° C. The optimum temperature and optimum pH and the reaction time depend on the polycarbamide and polycarbamate as well as the target extent of polymerization. During the condensation reaction, the resin-rich phase can start to separate from the water-rich phase. Most often, the condensation reaction is ended before such a separation occurs or, if more advanced resin is needed, water is distilled off from the separated resin mixtures to obtain homogeneous reaction products. After the target extent of poly-condensation is attained by monitoring the viscosity or other variables, the reaction is ended by adjusting the reaction mixture to pH 3.5-9.0 by adding 0.8% sodium hydroxide solution or other dilute bases and cooling to room temperature. The analyses of these advanced resins using ¹³C NMR spectroscopy showed various extents of formation of methylene and methylene-ether groups between amide or carbamate groups from some of the hydroxymethyl groups formed in the first alkaline step. A typical ¹³C NMR spectrum in FIG. 1 is shown for the resin GCAF of Example 1b with assigned carbon groups appearing over the chemical shift range of about 47˜160 ppm. Overall, the resin synthesis reaction patterns in the alkaline and acidic pH and the ¹³C NMR analysis results all agree with the chemical principles of typical thermosetting resins such as urea-formaldehyde resins, as illustrated below for glycerol tricarbamate-formaldehyde (GCAF) condensation resins:

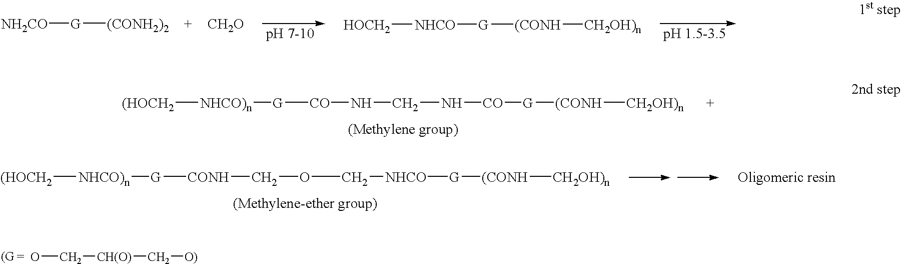

Syntheses of Copolymer Resins of Polycarbamide-Formaldehyde (CIF) or Polycarbamate-Formaldehyde (CAF) Resins with Urea

The polycarbamides and polycarbamates and their formaldehyde reaction products of the present invention are very well miscible with and react to form co-polymers with urea or urea-formaldehyde (UF) resins under the common resin synthesis and curing conditions. Many different mixing procedures can be used: urea or UF resins or UF concentrates can be added to finished CIF or CAF resins or in the beginning of synthesis procedures of CIF or CAF resins. Also, CIF resins, CAF resins, polycarbamates, or polycarbamides can be added to finished UF resins. For lowering the resin cost and various other reasons, these copolymers can be advantageous in various applications.

### Other Handling and Use Properties of Polycarbamide-Formaldehyde (CIF) and Polycarbamate-Formaldehyde (CAF) Resins of the Present Invention

The cooled, neutralized resins of the present invention can be stored or transported to the point of use and an acid catalyst is needed in application for accelerating the cure of resins when used as adhesives and laminates and the like. Strong acid catalysts such as sulfuric acid, hydrochloric acid, nitric acid, phosphoric acid, toluene sulfonic acid, formic acid and acid salts such as ammonium sulfate, ammonium chloride, ammonium phosphate, and any other strong acid salts of organic tertiary amines may be used in an amount of 0.1%-6.0% based on the weights of resin solids. In the case of bonding wood, no catalyst may be necessary due to the acids in the wood. The catalysts start the poly-condensation reaction of hydroxymethyl groups and external heating accelerates the curing reaction further. The hydroxymethyl groups react further with each other and also with the other carbamate or carbamide groups, so that the polymer molecules grow three-dimensionally and finally cross-link to form solid thermoset polymers of the adhesives, matrices, and the like.

Polycarbamide-formaldehyde and polycarbamate-formaldehyde resins are dispersible in water and therefore can be diluted by adding water or condensed by distillation of water or spray-dried to solid particles before application. In addition to curing catalysts, other agents can be added to the resins for other purposes: various ionic or non-ionic surfactants, water-miscible solvents such as methanol, ethanol, propanol, and the like, other thermosetting resins and materials such as urea, melamine, urea-formaldehyde resins, melamine-formaldehyde resins, urea-melamine-formaldehyde resins, phenol-formaldehyde resins, phenol-resorcinol-formaldehyde resins, and a variety of fillers such as wood floor, glass fiber, calcium carbonate, talc, celite, and the like, and a variety of pigments for coloring the cured resin materials. The aqueous resin compositions of the present invention may be dried at low temperatures, such as by spray-drying, and the solid resins powdered or granulated with or without fillers and the resulting mixture used as injection or compression molding.

### Use of Polycarbamide or Polycarbamate as Partial Replacement of Urea in Common Urea-Formaldehyde Resins

Since the polycarbamides (PCI) and polycarbamates (PCA) of the present invention react with formaldehyde very similarly as with urea, they can be incorporated in urea-formaldehyde (UF) resin in many ways, partially replacing the urea component. UF resins are well-known for various wood and other binder uses and their preparation method is also well-known (M. G. Kim and L. W. Amos, Industrial & Engineering Research, 29, 208 (1990)). In this well-known process, the first urea (U₁) and formaldehyde (F) are reacted at a F/U₁ mole ratio of between 1.8 and 2.4 under a weakly alkaline pH and at 90° C.˜100° C. for about 30 min. Then, the reaction mixture is acidified to weakly acidic pH and reacted until the target polymerization extent is reached, followed by adjusting the pH back to a weakly alkaline side. After cooling the reaction mixture to about 60° C., the second urea (U₂) is added and mixed and the finished resin is cooled to room temperature. The final F/(U+U) mole ratio depends on the amount of second urea, commonly reaching about 1.15 for particleboard binder applications. Thus, in this known procedure, PCI and PCA can be used to partially replace first urea, second urea, or both. Such resins can be made with up to 50% replacement of total urea by PCI or PCA and still have good handling characteristics and bonding performance.

### Testing and Evaluation Methods of Polycarbamide-Formaldehyde (CIF) or Polycarbamate-Formaldehyde (CAF) and Copolymer Resins

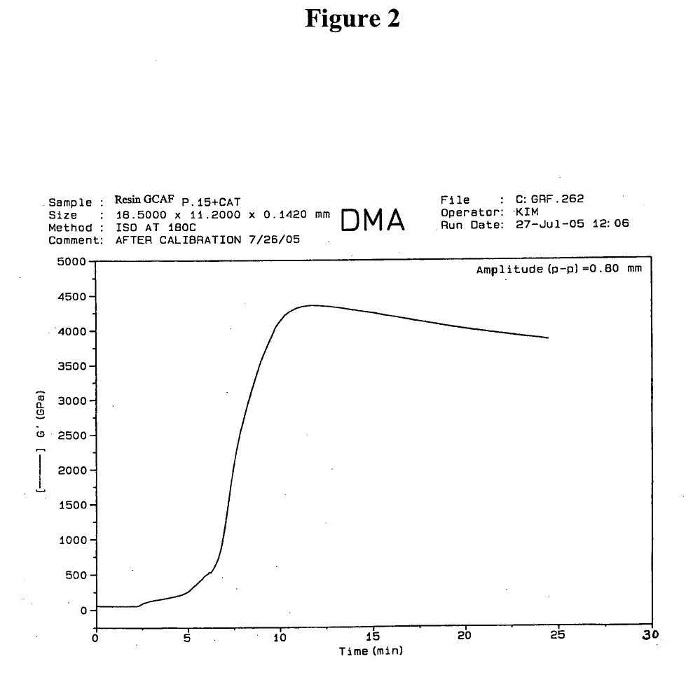

Dynamic mechanical analyzer (DMA) is a method widely used to measure and evaluate the curing process of thermosetting resins and their cured products (Lofthouse, M. G. and P. Burroughs, Journal Thermal Analysis 13, 439-453 (1978)). This method was used in the present invention using a DMA 983 from TA Instruments. FIG. 2 shows the DMA curing results of the resin GCAF of Example 1b of the present invention run with the sample heated at a rate of 25° C. per minute from room temperature to 180° C. and then isothermal until 25 minutes, showing the development of the rigidity of the sample (G′). In this procedure, a given amount of resin is impregnated into a piece of glass cloth (1.25 mm wide×18.5 mm long×0.15 mm thick) and the resin-impregnated glass cloth is clamped between the two arms of the instrument. When the test is started, the two arms are periodically flexing and the sample chamber heated according to a predetermined schedule. The sample's rigidity (shear modulus or strength) arising from thermosetting curing of resin is monitored. In all examples of resin curing reported in this disclosure, the chamber is heated from room temperature at a rate of 25° C. per min to a curing temperature of 160° C., 180° C., or 200° C. and then maintained at the final temperature (isothermal curing) for about 25 min. The resin-impregnated glass cloth starts from near zero strength and reaches the maximum strength after curing. The cure time of resin is defined as the time to reach the maximum strength. The maximum strength attained often degrades to a lower value due to heat-degradation for some resins in the later part of the test run, reflecting the (in)stability of the cured polymer backbone structures. In this measurement, after the sample is cured, the sample thickness value of cured resin sample is measured manually and incorporated into calculating the actual shear modulus values based on the final sample dimensions to compensate for small differences in resin weights loaded on samples. All synthesized resins in the Examples below were evaluated by this procedure at various temperatures. The maximum strength and cure time values and the stability of the cured resin obtained from DMA measurements, although they are relative (not absolute) values, allowed differentiation among different resins in curing performance. The maximum strength (rigidity) values are especially useful for comparing the soundness of cured polymer structures or the relative ranking of cross-link density values. Overall, all DMA data obtained amply demonstrated that resins of the present invention are truly thermosetting resins, agreeing with the chemical principles uncovered through resin synthesis and ¹³C NMR analysis results and are also favorably comparable with typical urea-formaldehyde resins.

¹³C NMR spectroscopy is an effective method of analyzing carbon chemical polymeric structures of polycarbamide-formaldehyde and polycarbamate-formaldehyde resins and copolymers used in the present invention, as shown by the example in FIG. 1. All starting materials and all synthesized resins of the Examples of this invention were analyzed by this method and the results of the chemical structures were in full agreement with those expected from the synthesis procedures and those presented in the present invention.

Laboratory board manufacture is another method often used for evaluating thermosetting wood adhesive resins in combination with testing for the internal bond strengths of boards. Particleboard is convenient to make in a laboratory, as well as medium density fiber board and hardwood plywood panels. Particleboards were made in the present invention using several selected resins in the Examples below, as follows: an amount of wood particles was weighed out to give a board 6 in.×6 in. square and 0.5 in. thick at a board density of 50 pounds per cubic feet; a catalyzed binder resin was sprayed onto the wood particles at resin solids level of 8.0% based on wood weight, and the ingredients were mixed well until a good dispersion of resin was attained; a uniform mat was made in a 6 in.×6 in. square box by dispersing and consolidating the resinated wood particles; the mat was transferred into a hot press pre-heated at the desired temperature; the mat was pressed to the target thickness in one min or so and kept closed for 3 min; the press was opened and the board cooled. Press temperatures were 160° C. for polycarbamide-formaldehyde and urea-formaldehyde resins and 210° C. for polycarbamate-formaldehyde resins. The board was then cut and tested for internal bond (IB) strength values according to the method of ASTM D1043.

The discussion and the description herein also present specific details to provide a thorough understanding of the present invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

### EXAMPLES

### Example 1a

### Glycerol Tricarbamate-Formaldehyde (GCAF) Resin

Thirty-three (33.0) grams of 50%-formaldehyde (F) solution (0.55 mole) kept at 60° C. and 32.0 grams of water were charged into a 200-ml reaction flask equipped with a cooling condenser, thermometer, stirrer, and heating mantle and the pH of the solution was adjusted to 8.5 by adding 8% sodium hydroxide solution. The formaldehyde solution was heated to about 80° C.-90° C. and 44.0 grams of glycerol tricarbamate (GCA) (0.20 mole) were added over a period of 20 min and the heating continued to maintain the reaction mixture at 90° C. for 10 min. The formaldehyde/glycerol tricarbamate (F/GCA) mole ratio reached 2.75. The reaction mixture became clear at the end of the 10 min heating period to indicate the dissolution of GCA from reaction with formaldehyde. The reaction was continued for 30 min at the same temperature with the pH of the reaction mixture maintained at 8.0. A small sample was taken and an analysis by using ¹³C NMR spectroscopy indicated the formation of hydroxymethyl groups nearly quantitatively. The results indicated the formation of hydroxymethyl groups bonded to carbamate groups of GCA. This product can be used without further reaction depending on application conditions.

### Example 1b

### Glycerol Tricarbamate-Formaldehyde (GCAF) Resin

The reaction product of Example 1a can be further condensed (or advanced) in degree of polymerization for uses under some application conditions. For this, the reaction product was acidified by adding 8% sulfuric acid solution to pH 2.0 and the temperature was raised to 95° C. The viscosity of the reaction mixture began at “A₁A” by the Gardener-Holdt Scale and the viscosity increased to “H” in 4 hours and 25 minutes. (In a duplicate cook, it was shown that a further cook beyond “H” viscosity resulted in separation of the resin-rich phase from the water-rich phase.) After the target “H” viscosity was attained, the condensation reaction was ended by adjusting the reaction mixture to pH 8.0 by adding 0.8% sodium hydroxide solution and cooling to room temperature. Drying of a one-gram sample of the GCAF resin at 125° C. for 2 hours resulted in 0.61 gram of colorless resin solids. The GCAF resin was analyzed using ¹³C NMR spectroscopy which indicated the formation of some methylene (46˜54 ppm) and methylene-ether (˜71 ppm) groups between GCA molecules from hydroxymethyl groups (˜63 ppm) formed in the alkaline step of Example 1a, shown as an example in FIG. 1. The GCAF resin was mixed with 0.5% ammonium sulfate catalyst based on the resin solids weight at room temperature and tested for curing using DMA at three different curing temperatures and the results were:

| | Cure temperature: | | |
|---|---|---|---|
| | 160° C. | 180° C. | 200° C. |
| Maximum shear modulus (psi): | 4400 | 4300 | 3700 |
| Cure time (min): | 13.5 | 9.5 | 7.5 |
| Heat stability: | Good | Good | Good |

The DMA results obtained at 180° C. are shown in FIG. 2 as an example, displaying the development of the sample's rigidity as the temperature of the sample was increased at a rate of 25° C. per min from room temperature to the final temperature and then holding at that temperature for about 25 min.

### Example 2

### meso-Erythritol Tetracarbamate-Formaldehyde (ECAF) Resin

Eight and four-tenths (8.40) grams of 50%-formaldehyde (F) solution (0.14 mole) kept at 60° C. and 15.0 grams of water were charged into a 200-ml reaction flask equipped with a cooling condenser, thermometer, stirrer, and heating mantle and the pH of the solution was adjusted to 8.5 by adding 8% sodium hydroxide solution. Then, the formaldehyde solution was heated to about 80° C.-90° C. and 10 grams (0.034 mole) of meso-erythritol tetracarbamate (ECA) were added in small portions over a period of 20 min. The F/ECA mole ratio reached 4.0. The heating continued at 90° C. The reaction mixture became clear after 30 min indicating the dissolution of ECA due to the reaction with formaldehyde. The reaction was continued for 3.0 hours at the same temperature with the pH of the reaction mixture maintained at 8.5. The reaction mixture was cooled and some water evaporated to yield 17.0 grams of the condensation products that showed resin-solids content of 82.0%. The sample analyzed using ¹³C NMR spectroscopy indicated the formation of hydroxymethyl groups bonded to the carbamate groups of ECA. The ECAF resin was mixed with 0.5% ammonium sulfate catalyst based on the resin solids weight at room temperature and tested for curing on DMA at three temperatures and the results were:

| | Cure temperature: | | |
|---|---|---|---|
| | 160° C. | 180° C. | 200° C. |
| Maximum shear modulus (psi): | 3350 | 3300 | 2750 |
| Cure time (min): | 11.0 | 9.5 | 7.5 |
| Heat stability: | Good | Good | Good |

### Example 3

### D-Xylitol Pentacarbamate-Formaldehyde (XCAF) Resin

Thirty (30.0) grams of 50%-formaldehyde (F) solution (0.50 mole) kept at 60° C. and 33.3 grams of water were charged into a 200-ml reaction flask equipped with a cooling condenser, thermometer, magnetic stirrer, and heating mantle and the pH of the solution was adjusted to 8.7 by adding 8% sodium hydroxide solution. Then, the formaldehyde solution was heated to about 80° C.-90° C. and 36.7 grams (0.10 mole) of D-xylitol pentacarbamate (XCA) were added in small portions over a period of 20 min. The F/XCA mole ratio reached 5.0. The heating of the reaction mixture was continued at 90° C.-95° C. The reaction mixture became clear after 35 min indicating the dissolution of XCA due to reaction with formaldehyde. The reaction was continued for 25 min at the same temperature with the pH of the reaction mixture maintained at 8.5. A small sample was taken and analyzed using ¹³C NMR spectroscopy which indicated the quantitative formation of hydroxymethyl groups bonded to the carbamate groups of XCA. Then, the reaction mixture was acidified by adding 8% sulfuric acid solution to pH 1.5 and the temperature was raised to 98° C. The viscosity of the reaction mixture began at “A₁A” by the Gardener-Holdt Scale and increased to “EF” after 40 min. (In a duplicate cook, a further cook resulted in a separation of the resin-rich phase from the water-rich phase.) After the “EF” viscosity was attained, the condensation reaction was ended by adjusting the reaction mixture to pH 8.0 by adding 0.8% sodium hydroxide solution and cooling to room temperature. Drying of a one-gram sample of the XCAF resin at 125° C. for 2 hours resulted in 0.51 gram of colorless resin solids (51% resin solids content). The XCAF resin was analyzed using ¹³C NMR spectroscopy which indicated the formation of methylene and methylene-ether bonds from hydroxymethyl groups. The XCAF resin was mixed with 0.5% ammonium sulfate catalyst based on the resin solids weight at room temperature and tested for curing using DMA at three different curing temperatures and the results were:

| | Cure temperature: | | |
|---|---|---|---|
| | 160° C. | 180° C. | 200° C. |
| Maximum shear modulus (psi): | 3100 | 3100 | 3950 |
| Cure time (min): | 8.5 | 7.4 | 6.5 |
| Heat stability: | Good | Good | Good |

### Example 4

### D-Sorbitol Hexacarbamate-Formaldehyde (SCAF) Resin

Eight and two-tenths (8.20) grams of 50%-formaldehyde (F) solution (0.137 mole) kept at 60° C. and 15.0 grams of water were charged into a 200-ml reaction flask equipped with a cooling condenser, thermometer, magnetic stirrer, and heating mantle and the pH of the solution was adjusted to 8.5 by adding 8% sodium hydroxide solution. Then, the formaldehyde solution was heated to about 80° C.-90° C. and 10 grams (0.023 mole) of D-sorbitol hexacarbamate (SCA) were added in small portions over a period of 20 min. The F/SCA mole ratio reached 6.0. The heating was continued to maintain the temperature at 90° C. The reaction mixture became clear after 30 min indicating the dissolution of SCA due to the reaction with formaldehyde. The reaction was continued for 3.0 hours at the same temperature with the pH of the reaction mixture maintained at 8.5. The reaction mixture was cooled to give SCAF condensation resin that showed resin solids content of 36.2%. The sample analyzed using ¹³C NMR spectroscopy indicated the quantitative formation of hydroxymethyl groups bonded to the carbamate groups of SCA molecules. The SCAF resin was mixed with 0.5% ammonium sulfate catalyst based on the resin solids weight at room temperature and tested for curing on DMA at two temperatures and the results were:

Cure temperature:

| | 180° C. | 200° C. |
|---|---|---|
| Maximum shear modulus (psi): | 4100 | 4600 |
| Cure time (min): | 9.0 | 6.5 |
| Heat stability: | Good | Medium |

### Example 5

### D-Mannitol Hexacarbamate-Formaldehyde (MCAF) Resin

Eight and two-tenths (8.20) grams of 50%-formaldehyde (F) solution (0.137 mole) kept at 60° C. and 15.0 grams of water were charged into a 200-ml reaction flask equipped with a cooling condenser, thermometer, magnetic stirrer, and heating mantle and the pH of the solution was adjusted to 8.5 by adding 8% sodium hydroxide solution. Then, the formaldehyde solution was heated to about 80° C.-90° C. and 10 grams (0.023 mole) of D-mannitol hexacarbamate (MCA) were added in small portions over a period of 20 min. The F/MCA mole ratio reached 4.0. The heating was continued to maintain the temperature at 90° C. The reaction mixture became clear after 30 min indicating the dissolution of MCA due to the reaction with formaldehyde. The reaction was continued for 3.0 hours at the same temperature with the pH of the reaction mixture maintained at 8.5. The reaction mixture was cooled to give MCAF condensation resin that showed resin solids content of 35.5%. The sample analyzed using ¹³C NMR spectroscopy indicated the quantitative formation of hydroxymethyl groups bonded to the carbamate groups of MCA molecules. The MCAF resin was mixed with 0.5% ammonium sulfate catalyst based on the resin solids weight at room temperature and tested for curing on DMA at one temperature and the results were:

| | Cure temperature: |
|---|---|
| | 160° C. |
| Maximum shear modulus (psi): | 2850 |
| Cure time (min): | 9.5 |
| Heat stability: | Good |

### Example 6

### 1,1,1-Trihydroxymethylethane Tricarbamate-Formaldehyde (TECAF) Resin

Twenty-eight (28.0) grams of 50%-formaldehyde (F) solution (0.47 mole) kept at 60° C. and 32.0 grams of water were charged into a 200-ml reaction flask equipped with a cooling condenser, thermometer, magnetic stirrer, and heating mantle and the pH of the solution was adjusted to 8.5 by adding 8% sodium hydroxide solution. Then, the formaldehyde solution was heated to about 70° C. and 40.0 grams of 1,1,1-trihydroxymethylethane tricarbamate (TECA) (0.16 mole) were added in small portions over a period of 20 min. The F/TECA mole ratio reached 3.0. The heating of the reaction mixture was continued to maintain the reaction mixture at 70° C.-85° C. and the reaction mixture became clear after 10 min period indicating the dissolution of TECA from the reaction with formaldehyde. The reaction was continued for 30 min at the same temperature with the pH of the reaction mixture maintained at 8.0. A small sample taken and analyzed using ¹³C NMR spectroscopy indicated the formation of hydroxymethyl groups bonded to the carbamate groups of TECA molecules. Then, the reaction mixture was acidified by adding 8% sulfuric acid solution to pH 1.8 and the temperature was raised to 95° C. The viscosity of the reaction mixture began at “A₁A” by the Gardener-Holdt Scale and increased to “B” after one hour. The poly-condensation reaction was ended by adjusting the reaction mixture to pH 7.0 by adding 0.8% sodium hydroxide solution and cooled to room temperature to give a TECAF resin. Drying of a one-gram sample of the TECAF resin at 125° C. for 2 hours resulted in 0.54 gram of colorless resin solids (54.0% resin solids content). The TECAF resin was analyzed using ¹³C NMR spectroscopy which indicated the formation of methylene and methylene-ether bonds as well as hydroxymethyl groups on carbamate groups of TECA molecules. The TECAF resin was mixed with 0.5% ammonium sulfate catalyst based on the resin solids weight at room temperature and tested for curing using DMA at three different curing temperatures and the results were:

| | Cure temperature: | | |
|---|---|---|---|
| | 160° C. | 180° C. | 200° C. |
| Maximum shear modulus | 4300 | 5100 | 4400 |
| (psi): | | | |
| Cure time (min): | 11.5 | 8.5 | 7.5 |
| Heat stability: | Good | Good | Medium |

### Example 7

### 1,1,1-Trihydroxymethylpropane Tricarbamate-Formaldehyde (TPCAF) Resin

Six and eight-tenths (6.80) grams of 50%-formaldehyde (F) solution (0.11 mole) kept at 60° C. were charged into a 50-ml reaction flask equipped with a cooling condenser, thermometer, magnetic stirrer, and heating mantle and the pH of the solution was adjusted to 8.5 by adding 8% sodium hydroxide solution. Then, the formaldehyde solution was heated to about 85° C.-90° C. and 10 grams (0.038 mole) grams of 1,1,1-trihydroxymethylpropane tricarbamate (TPCA) were added in small portions over a period of 20 min. The F/TPCA mole ratio reached 3.0. The heating was continued to maintain the temperature at 85° C. The reaction mixture became clear after 20 min indicating the dissolution of TPCA due to the reaction with formaldehyde. The reaction was continued for one hour at the same temperature with the pH of the reaction mixture maintained at 8.0. Approximately five (5.0) grams of water was evaporated from the reaction mixture which was then cooled to give a TPCAF resin that showed resin solids content of 76.0%. The sample analyzed using ¹³C NMR spectroscopy indicated the formation of hydroxymethyl groups bonded to the carbamate groups of TPCA. The TPCAF resin was mixed with 0.5% ammonium sulfate catalyst based on the resin solids weight at room temperature and tested for curing on DMA at three temperatures and the results were:

| | Cure temperature: | | |
|---|---|---|---|
| | 160° C. | 180° C. | 200° C. |
| Maximum shear modulus | 3700 | 3300 | 3300 |
| (psi): | | | |
| Cure time (min): | 14.0 | 11.0 | 5.5 |
| Heat stability: | Good | Good | Good |

### Example 8

### Pentaerythritol Tetracarbamate-Formaldehyde (PCAF) Resin

Forty-two (42.0) grams of 50%-formaldehyde (F) solution (0.70 mole) kept at 60° C. and 30.0 grams of water were charged into a 200-ml reaction flask equipped with a cooling condenser, thermometer, magnetic stirrer, and heating mantle and the pH of the solution was adjusted to 8.5 by adding 8% sodium hydroxide solution. Then, the formaldehyde solution was heated to about 80° C.-90° C. and 61.6 grams of pentaerythritol tetracarbamate (PCA) (0.20 mole) were added in small portions over a period of 20 min. The F/PCA mole ratio reached 3.50. The heating of the reaction mixture was continued to maintain the reaction mixture at 80° C.-95° C. and the reaction mixture became clear at the end of an additional period of time indicating the dissolution of PCA from the reaction with formaldehyde. The reaction was continued for 140 min at the same temperature with the pH of the reaction mixture maintained at 8.0. A small sample taken and analyzed using ¹³C NMR spectroscopy indicated the formation of hydroxymethyl groups bonded to carbamate groups of PCA molecules. Then, the reaction mixture was acidified by adding 8% sulfuric acid solution to pH 2.8 and the temperature was raised to 95° C. The viscosity of the reaction mixture began at “A₁A” by the Gardener-Holdt Scale and increased to “G” after 3.0 hours. The poly-condensation reaction was ended by adjusting the reaction mixture to pH 5.5 by adding 0.8% sodium hydroxide solution and cooling to room temperature, resulting in a PCAF resin. Drying of a one-gram sample of the PCAF resin at 125° C. for 2 hours resulted in 0.60 gram of colorless resin solids (60.0% resin solids content). The PCAF resin was analyzed using ¹³C NMR spectroscopy which indicated the formation of methylene and methylene-ether bonds from hydroxymethyl groups on carbamate groups. The PCAF resin was mixed with 0.5% ammonium sulfate catalyst based on the resin solids weight at room temperature and tested for curing using DMA at three different curing temperatures and the results were:

| | Cure temperature: | | |
|---|---|---|---|
| | 160° C. | 180° C. | 200° C. |
| Maximum shear modulus | 4800 | 4250 | 4550 |
| (psi): | | | |
| Cure time (min): | 12.5 | 9.5 | 8.0 |
| Heat stability: | Good | Good | Good |

### Example 9

### Ethylene Glycol Dicarbamate-Formaldehyde (EGCAF) Resin

Forty three (43.0) grams of 50%-formaldehyde (F) solution (0.71 mole) kept at 60° C. and 40.0 grams of water were charged into a 200-ml reaction flask equipped with a cooling condenser, thermometer, magnetic stirrer, and heating mantle and the pH of the solution was adjusted to 9.0 by adding 8% sodium hydroxide solution. Then, the formaldehyde solution was heated to about 85° C. and 60.0 grams of ethylene glycol dicarbamate (EGCA) (0.40 mole) were added in small portions over a period of 20 min. The F/EGCA mole ratio reached 1.80. The heating of the reaction mixture was continued to maintain the reaction mixture at 85° C.-93° C. and the reaction mixture became clear after a 10 min period indicating the dissolution of EGCA from the reaction with formaldehyde. The reaction was continued for 70 min at the same temperature with the pH of the reaction mixture maintained at 8.0. A sample taken and analyzed using ¹³C NMR spectroscopy indicated the quantitative formation of hydroxymethyl groups bonded to carbamate groups of EGCA molecules. The reaction mixture was then acidified by adding 8% sulfuric acid solution to pH 2.5 and the temperature was raised to 93° C. for one hour. The viscosity of the reaction mixture began at “A₁A” by the Gardener-Holdt Scale and increased to “A.” The polycondensation reaction was ended by adjusting the reaction mixture to pH 5.5 by adding 0.8% sodium hydroxide solution and cooling to room temperature. Drying of a one-gram sample of the EGCAF resin at 125° C. for 2 hours resulted in 0.55 gram of colorless resin solids (55.0 resin solids content). The EGCAF resin was analyzed using ¹³C NMR spectroscopy which indicated the formation of methylene and methylene-ether bonds from hydroxymethyl groups on carbamate groups. The EGCAF resin was mixed with 0.5% ammonium sulfate catalyst based on the resin solids weight at room temperature and tested for curing using DMA at three different curing temperatures and the results were:

| | Cure temperature: | | |
|---|---|---|---|
| | 160° C. | 180° C. | 200° C. |
| Maximum shear modulus | 1800 | 1750 | 2240 |
| (psi): | | | |
| Cure time (min): | 13.0 | 8.0 | 7.4 |
| Heat stability: | Good | Good | Good |

### Example 10

As an example to show the utility of mixing different polycarbamate-formaldehyde resins, the EGCAF resin of Example 9 was mixed with PCAF resin of Example 8 in a 1:2 ratio (by weight) and mixed with 0.5% ammonium sulfate catalyst based on the resin solids weight at room temperature and tested for curing using DMA and the results were:

| | Cure temperature: |
|---|---|
| | 180° C. |
| Maximum shear modulus (psi): | 3050 |
| Cure time (min): | 8.5 |
| Heat stability: | Good |

### Example 11

As an example to show the utility of mixing polycarbamate-formaldehyde resins with urea (U), the GCAF resin of Example 1b was mixed with urea in an amount to reduce the molar ratio to a combined F/(GCA+U)=2.0 and then mixed with 0.5% ammonium sulfate catalyst based on the resin solids weight at room temperature and tested for curing using DMA and the results were:

| | Cure temperature: |
|---|---|
| | 160° C. |
| Maximum shear modulus (psi): | 3550 |
| Cure time (min): | 11.5 |
| Heat stability: | Good |

### Example 12

As an example to show the utility of mixing different polycarbamate-formaldehyde resins, the GCAF resin of Example 1b was mixed with the MCAF resin of Example 5 in a 1:4 ratio and then mixed with 0.5% ammonium sulfate catalyst based on the resin solids weight at room temperature and tested for curing using DMA and the results were:

| | Cure temperature: |
|---|---|
| | 160° C. |
| Maximum shear modulus (psi): | 3150 |
| Cure time (min): | 9.2 |
| Heat stability: | Good |

### Example 13

As another example to show the utility of mixing different polycarbamate-formaldehyde resins, the SCAF resin of Example 4 and the PCAF resin of Example 8 were mixed in a 1:1 ratio and then mixed with 0.5% ammonium sulfate catalyst based on the resin solids weight at room temperature and tested for curing using DMA and the results were:

| | Cure temperature: |
|---|---|
| | 160° C. |
| Maximum shear modulus (psi): | 4600 |
| Cure time (min): | 8.5 |
| Heat stability: | Good |

### Example 14

### Ethylene Dicarbamide-Formaldehyde (EDCIF) Resin

One hundred twenty-three (123.0) grams of 50%-formaldehyde (F) solution (2.05 moles) kept at 60° C. and 40.0 grams of water were charged into a 500 mL reaction flask equipped with a cooling condenser, thermometer, magnetic stirrer, and heating mantle and the pH of the solution was adjusted to 8.0 by adding 8% sodium hydroxide solution. Then, the formaldehyde solution was heated to about 85° C. and 200.0 grams of ethylene dicarbamide (EDCI) (1.37 moles) were added in small portions over a period of 20 min. The F/EDCI mole ratio reached 1.50. The heating of the reaction mixture was continued to maintain the reaction mixture at 85° C.-93° C. and the reaction mixture became clear after a 10 min period indicating the dissolution of EDCI from the reaction with formaldehyde. The reaction was continued for 30 min at the same temperature with the pH of the reaction mixture maintained at 8.0. A sample taken and analyzed using ¹³C NMR spectroscopy indicated the quantitative formation of hydroxymethyl groups bonded to the carbamide groups of EDCI molecules. The reaction mixture was then acidified by adding 8% sulfuric acid solution to pH 5.0 and the temperature was maintained at 75° C. for one hour. The viscosity of the reaction mixture began at “A₁A” by the Gardener-Holdt Scale and increased to “JK.” The polycondensation reaction was ended by adjusting the reaction mixture to pH 8.0 by adding 0.8% sodium hydroxide solution and cooling to room temperature. Drying of a one-gram sample of the EDCIF resin at 125° C. for 2 hours resulted in 0.55 gram of colorless resin solids (55.0% resin solids content). The EDCIF resin was analyzed using ¹³C NMR spectroscopy which indicated the formation of methylene and methylene-ether bonds as well as hydroxymethyl groups on carbamide groups of EDCI. The EDCIF resin was mixed with 0.5% ammonium sulfate catalyst based on the resin solids weight at room temperature and tested for curing using DMA at 160° C. and the results were:

| | Cure temperature: |
|---|---|
| | 160° C. |
| Maximum shear modulus (psi): | 1800 |
| Cure time (min): | 7.1 |
| Heat stability: | Good |

### Example 15

### Diethylene Tricarbamide-Formaldehyde (DTCIF) Resin

One hundred twenty (120.0) grams of 50%-formaldehyde (F) solution (2.0 moles) kept at 60° C. and 100.0 grams of water were charged into a 500 mL reaction flask equipped with a cooling condenser, thermometer, magnetic stirrer, and heating mantle and the pH of the solution was adjusted to 8.5 by adding 8% sodium hydroxide solution. Then, the formaldehyde solution was heated to about 85° C. and 232.0 grams of diethylene tricarbamide (DTCI) (1.0 mole) were added in small portions over a period of 20 min. The F/DTCI mole ratio reached 2.0. The heating of the reaction mixture was continued to maintain the reaction mixture at 85° C.-93° C. and the reaction mixture became clear after a 10 min period indicating the dissolution of DTCI from the reaction with formaldehyde. The reaction was continued for 30 min at the same temperature with the pH of the reaction mixture maintained at 8.0. A sample taken and analyzed using ¹³C NMR spectroscopy indicated the quantitative formation of hydroxymethyl groups bonded to the carbamide groups of DTCI molecules. The reaction mixture was then acidified by adding 8% sulfuric acid solution to pH 5.0 and the temperature was maintained at 75° C. for one hour. The viscosity of the reaction mixture began at “A” by the Gardener-Holdt Scale and increased to “R.” The polycondensation reaction was ended by adjusting the reaction mixture to pH 8.0 by adding 0.8% sodium hydroxide solution and cooling to room temperature. Drying of a one-gram sample of the DTCIF resin at 125° C. for 2 hours resulted in 0.55 gram of colorless resin solids (55.0% resin solids content). The DTCIF resin was analyzed using ¹³C NMR spectroscopy which indicated the formation of methylene and methylene-ether bonds as well as hydroxymethyl groups on the carbamide groups of DTCI. The DTCIF resin was mixed with 0.5% ammonium sulfate catalyst based on the resin solids weight at room temperature and tested for curing using DMA at 160° C. and the results were:

| | Cure temperature: |
|---|---|
| | 160° C. |
| Maximum shear modulus (psi): | 2700 |
| Cure time (min): | 7.0 |
| Heat stability: | Good |

### Example 16

### Mixing Polycarbamide-Formaldehyde Resins with Urea

As an example to show the utility of adding urea to polycarbamide-formaldehyde resins, a batch of DTCIF resin of Example 15 was made and mixed with 19.8 grams of urea (U), resulting in a resin with F/(DTCI+U) mole ratio of 1.50. This resin was then mixed with 0.5% ammonium sulfate catalyst based on the resin solids weight at room temperature and tested for curing using DMA at 160° C. and the results were:

| | Cure temperature: |
|---|---|
| | 160° C. |
| Maximum shear modulus (psi): | 2300 |
| Cure time (min): | 7.1 |
| Heat stability: | Good |

### Example 17

### Mixing Two or More Different Polycarbamide-Formaldehyde Resins

As an example to show the utility of mixing a polycarbamide-formaldehyde resin with another, EDCIF resin of Example 14 and DTCIF resin of Example 15 were mixed in a 1:1 ratio and then mixed with 0.5% ammonium sulfate catalyst based on the resin solids weight at room temperature and tested for curing using DMA at 160° C. and the results were:

| | Cure temperature: |
|---|---|
| | 160° C. |
| Maximum shear modulus (psi): | 2200 |
| Cure time (min): | 7.0 |
| Heat stability: | Good |

### Example 18

### A Typical Urea-Formaldehyde (UF) Resin for Comparative Purposes

By using a commercial-grade urea and a 50% formaldehyde solution, a typical UF resin was prepared as follows: 300.0 grams of 50% formaldehyde solution (5.0 moles) were charged into a stirred reactor, the pH adjusted to 8.0 with an 8% sodium hydroxide solution, and the reactor heated to 70° C. Then, 143 grams of urea (first urea) were added over a period of 20 min while the reaction exotherm and heating control were used to raise the temperature to 90° C. The reaction temperature was maintained by intermittent cooling and, later, by heating for 30 min. Then, by using 8% sulfuric acid solution the pH was lowered to 5.0-5.1 and, by heating, the temperature raised to 95° C. The reaction mixture was kept under this condition for about 110 min with the viscosity advancing to “T” by the Gardner-Holdt Scale. Then, the pH of the reaction mixture was adjusted using 8% sodium hydroxide solution to 8.0 and cooling applied to reach about 60° C., when 118 grams of urea (second urea) were added and stirred until the resin cooled to room temperature. The resin had a viscosity of “K” by the Gardener-Holdt Scale and solids content of 62.5% with calculated formaldehyde/urea mole ratio of 1.15, typical values of current industrial UF resins used in particleboard manufacturing. This resin was mixed with 0.5% ammonium sulfate catalyst based on the resin solids weight at room temperature and tested for curing using DMA at 160° C. and the results were:

| | Cure temperature: |
|---|---|
| | 160° C. |
| Maximum shear modulus (psi): | 1850 |
| Cure time (min): | 7.1 |
| Heat stability: | Good |

### Example 19

### A Typical Example using a Polycarbamide as a Partial Replacement of Urea in Urea-Formaldehyde (UF) Resins

Ethylene dicarbamide (EDCI) was used as an example. The first part of Example 18 was repeated until a slightly lower viscosity target was reached as follows: 300.0 grams of 50% formaldehyde solution (5.0 moles) were charged into a stirred reactor, the pH adjusted to 8.0 with an 8% sodium hydroxide solution, and the reactor heated to 70° C. Then, 143 grams of urea (first urea) were added over a period of 20 min while the reaction exotherm and heating control were used to raise the temperature to 90° C. The reaction temperature was maintained then by intermittent cooling and, later, by heating for 30 min and then, by using 8% sulfuric acid solution, the pH was lowered to 5.0-5.1 and, by heating, the temperature raised to 95° C. The reaction mixture was kept under this condition for about 110 min with the viscosity advancing to “OP” by Gardner-Holdt Scale and then the pH of the reaction mixture was adjusted using 8% sodium hydroxide solution to 8.0 and cooling applied to reach about 80° C. Then, 138.7 grams of ethylene dicarbamide (0.95 mole) were added and stirred until the resin cooled to room temperature. The resin had a viscosity of “L” by the Gardener-Holdt Scale and solids content of 61.2% with calculated F/(U+EDCI) mole ratio of 1.50. This resin was mixed with 0.5% ammonium sulfate catalyst based on the resin solids weight at room temperature and tested for curing using DMA at 160° C. and the results were:

| | Cure temperature: |
|---|---|
| | 160° C. |
| Maximum shear modulus (psi): | 2250 |
| Cure time (min): | 7.0 |
| Heat stability: | Good |

### Example 20

### Bonding Particleboard using Various Resins of this Disclosure and a Comparative Urea-Formaldehyde Resin and Testing of Particleboard

Laboratory particleboards were manufactured using several selected resins from the Examples shown above using common current procedures and parameters used by the particleboard industry as follows: board dimensions of 6″×6″ and 0.5″ thickness; target board density of 50 pounds per cubic feet; binder resin loading level of 10.0% based on wood weight; press time of 4.0 min including one min press-closing time; and press temperatures of 160° C. for polycarbamide-formaldehyde and urea-formaldehyde resins and 210° C. for polycarbamate-formaldehyde resins. The internal bond (IB) strength values were obtained for these boards according to the method described in ASTM D1043, as follows:

| | Example | | | | | | |
|---|---|---|---|---|---|---|---|
| | No. 1b | No. 8 | No. 11 | No. 15 | No. 16 | No. 17 | No. 18 |
| Resin | GCAF | PCAF | GCAF + U | DTCIF | DTCIF + U | EDCIF + DTCIF | UF |
| acronym | | | | | | | |
| IB strength | 181 | 210 | 163 | 161 | 150 | 152 | 150 |
| (psi) | | | | | | | |

In conclusion, board making and test results in the Examples have demonstrated that polycarbamate-formaldehyde and polycarbamide-formaldehyde resins of the present invention are truly thermosetting resins capable of producing strong structural polymer materials useful in many applications, exemplified in bonding of wood particle board using current manufacturing processes. This disclosure has for the first time described and fully characterized the synthesis procedures and structural identities of polycarbamides and polycarbamates and their formaldehyde reaction products. Moreover, this disclosure shows their usefulness in various applications.

The above is a detailed description of particular embodiments of the present invention. All embodiments disclosed and claimed herein can be easily executed in light of this disclosure. While various embodiments of the present invention have been described above, it should be understood that they have been presented by way of example and not limitation. Those of ordinary skill in the relevant art(s), in light of the present disclosure, should recognize and understand that a wide variety of various and obvious changes, alternatives, variations, and modifications in form and detail of the embodiments disclosed herein can be selected and made therein without departing from the true scope and spirit of the present invention. After reading the above description, it will be apparent to those skilled in the relevant art(s) how to implement the invention in alternative embodiments. Thus, the present invention should not be limited by any of the above-described exemplary embodiments. The invention is described both generically and regarding specific embodiments, while the full scope of the invention is set out in the claims and their equivalents that follow. The disclosure and description presented further explain the invention and are not to be interpreted or inferred as limiting thereof. The claims and specification should not be construed to unduly narrow the complete scope of protection to which the present invention is entitled. The disclosure and appended claims are intended to cover all modifications that may fall within the scope of the claims.

Moreover, the present invention is complex in nature and is generally best practiced by empirically determining the appropriate values of the operating parameters, or by conducting computer simulations, to arrive at best design for a given application. Accordingly, all suitable modifications, combinations, and equivalents should be considered as falling within the spirit and scope of the invention. It should also be understood that the figures are presented for example purposes only.

The purpose of the abstract of the disclosure is to enable the U.S. Patent and Trademark Office, the public in general, and particularly the scientists, engineers, and practitioners in the art who are unfamiliar with patent or legal terms or phraseology, to efficiently determine from a cursory inspection the nature and essence of the technical disclosure of the application. The abstract of the disclosure is therefore not intended in any way to be limiting as to the scope of the present invention.

What is claimed is:

1. A compound of the general formula with a common name of glycerol tricarbamate:

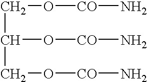

2. A compound of the general formula with a common name of meso-erythritol tetracarbamate:

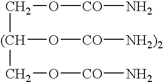

3. A compound of the general formula with a common name of D-xylitol pentacarbamate:

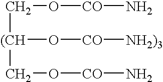

4. A compound of the general formula with a common name of D-sorbitol hexacarbamate:

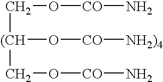

5. A compound of the general formula with a common name of D-mannitol hexacarbamate:

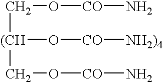

6. A compound of the general formula with a common name of 1,1,1-trihydroxymethyl ethane tricarbamate:
CH₃C—(CH₂—O—CO—NH₂)₃

7. A compound of the general formula with a common name of 1,1,1-trihydroxymethyl propane tricarbamate:
CH₃CH₂—C—(CH₂—O—CO—NH₂)₃

8. A compound of the general formula with a common name of pentaerythritol tetracarbamate:
C—(CH₂—O—CO—NH₂)₄

9. A compound of the general formula with a common name of diethylene tricarbamide:
NH₂CONHCH₂—CH₂N(CONH₂)CH₂—CH₂NHCONH₂

10. A process for preparing glycerol tricarbamate comprising the steps of: charging and cooling tetrahydrofuran in a reactor, condensing phosgene in said reactor to form a first solution, adding a catalytic amount of pyridine to said first solution to form a second solution, adding glycerol to said second solution to form a solution of glycerol trichloroformate, reacting said solution of glycerol trichloroformate at a low temperature by introducing ammonia or a solution of saturated ammonium hydroxide to form glycerol tricarbamate crystals, collecting the glycerol tricarbamate crystals by filtration and drying the crystals; and optionally re-crystallizing the glycerol tricarbamate crystals in water or any organic solvent.

11. A process for preparing meso-erythritol tetracarbamate comprising the steps of: charging and cooling tetrahydrofuran in a reactor, condensing phosgene in said reactor to form a first solution, adding a catalytic amount of pyridine to said first solution to form a second solution, adding meso-erythritol to said second solution to form a solution of meso-erythritol trichloroformate, reacting said solution of meso-erythritol trichloroformate at a low temperature by introducing ammonia or a solution of saturated ammonium hydroxide to form meso-erythritol tetracarbamate crystals, collecting the meso-erythritol tetracarbamate crystals by filtration and drying the crystals; and optionally re-crystallizing the meso-erythritol tetracarbamate crystals in water or any organic solvent.

12. A process for preparing D-xylitol pentacarbamate comprising the steps of: charging and cooling tetrahydrofuran in a reactor, condensing phosgene in said reactor to form a first solution, adding a catalytic amount of pyridine to said first solution to form a second solution, adding D-xylitol to said second solution to form a solution of D-xylitol trichloroformate, reacting said solution of D-xylitol trichloroformate at a low temperature by introducing ammonia or a solution of saturated ammonium hydroxide to form D-xylitol pentacarbamate crystals, collecting the D-xylitol pentacarbamate crystals by filtration and drying the crystals; and optionally re-crystallizing the D-xylitol pentacarbamate crystals in water or any organic solvent.

13. A process for preparing D-sorbitol hexacarbamate comprising the steps of: charging and cooling tetrahydrofuran in a reactor, condensing phosgene in said reactor to form a first solution, adding a catalytic amount of pyridine to said first solution to form a second solution, adding D-sorbitol to said second solution to form a solution of D-sorbitol trichloroformate, reacting said solution of D-sorbitol trichloroformate at a low temperature by introducing ammonia or a solution of saturated ammonium hydroxide to form D-sorbitol hexacarbamate crystals, collecting the D-sorbitol hexacarbamate crystals by filtration and drying the crystals; and optionally re-crystallizing the D-sorbitol hexacarbamate crystals in water or any organic solvent.

14. A process for preparing D-mannitol hexacarbamate comprising the steps of: charging and cooling tetrahydrofuran in a reactor, condensing phosgene in said reactor to form a first solution, adding a catalytic amount of pyridine to said first solution to form a second solution, adding D-mannitol to said second solution to form a solution of D-mannitol trichloroformate, reacting said solution of D-mannitol trichloroformate at a low temperature by introducing ammonia or a solution of saturated ammonium hydroxide to form D-mannitol hexacarbamate crystals, collecting the D-mannitol hexacarbamate crystals by filtration and drying the crystals; and optionally re-crystallizing the D-mannitol hexacarbamate crystals in water or any organic solvent.

15. A process for preparing 1,1,1-trihydroxymethylethane tricarbamate comprising the steps of: charging and cooling tetrahydrofuran in a reactor, condensing phosgene in said reactor to form a first solution, adding a catalytic amount of pyridine to said first solution to form a second solution, adding 1,1,1-trihydroxymethylethane to said second solution to form a solution of 1,1,1-trihydroxymethylethane trichloroformate, reacting said solution of 1,1,1-trihydroxymethylethane trichloroformate at a low temperature by introducing ammonia or a solution of saturated ammonium hydroxide to form 1,1,1-trihydroxymethylethane tricarbamate crystals, collecting the 1,1,1-trihydroxymethylethane tricarbamate crystals by filtration and drying the crystals; and optionally re-crystallizing the 1,1,1-trihydroxymethylethane tricarbamate crystals in water or any organic solvent.

16. A process for preparing 1,1,1-trihydroxymethylpropane tricarbamate comprising the steps of: charging and cooling tetrahydrofuran in a reactor, condensing phosgene in said reactor to form a first solution, adding a catalytic amount of pyridine to said first solution to form a second solution, adding 1,1,1-trihydroxymethylpropane to said second solution to form a solution of 1,1,1-trihydroxymethylpropane trichloroformate, reacting said solution of 1,1,1-trihydroxymethylpropane trichloroformate at a low temperature by introducing ammonia or a solution of saturated ammonium hydroxide to form 1,1,1-trihydroxymethylpropane tricarbamate crystals, collecting the 1,1,1-trihydroxymethylpropane tricarbamate crystals by filtration and drying the crystals; and optionally re-crystallizing the 1,1,1-trihydroxymethylpropane tricarbamate crystals in water or any organic solvent.

17. A process for preparing pentaerythritol tetracarbamate comprising the steps of: charging and cooling tetrahydrofuran in a reactor, condensing phosgene in said reactor to form a first solution, adding a catalytic amount of pyridine to said first solution to form a second solution, adding pentaerythritol to said second solution to form a solution of pentaerythritol trichloroformate, reacting said solution of pentaerythritol trichloroformate at a low temperature by introducing ammonia or a solution of saturated ammonium hydroxide to form pentaerythritol tetracarbamate crystals, collecting the pentaerythritol tetracarbamate crystals by filtration and drying the crystals; and optionally re-crystallizing the pentaerythritol tetracarbamate crystals in water or any organic solvent.

18. A process for preparing diethylene tricarbamide comprising the steps of: mixing diethylenetriamine with water and urea, reacting said mixture by heating to generate an ammonia by-product, removing the ammonia by distillation with water to form a first solution, cooling said first solution to form precipitates which are collected as crude diethylene tricarbamide; and optionally purifying the crude diethylene tricarbamide by dissolving in water at an elevated temperature to form a second solution, cooling said second solution to form diethylene tricarbamide crystals, and collecting the diethylene tricarbamide crystals by filtration and drying the crystals; and optionally washing the wet diethylene tricarbamide crystals with ethanol and drying the crystals to obtain pure diethylene tricarbamide.

19. A process for preparing diethylene tricarbamide comprising the steps of: mixing ethylenediamine and an equivalent amount of acid in water, reacting said mixture by heating at a temperature of 50° C. to 90° C. to form a first solution, adding sodium cyanate to said first solution, cooling said first solution to form diethylene tricarbamide crystals, and collecting the diethylene tricarbamide crystals by filtration and drying the crystals; and optionally washing the wet diethylene tricarbamide crystals with ethanol and drying the crystals to obtain pure diethylene tricarbamide.

20. A thermosetting resin composition comprising a 0.5%-100.0% aqueous dispersion or solids, or combination thereof, made by reacting a mixture comprising glycerol tricarbamate and formaldehyde to form a condensate, in which the molar ratio of formaldehyde to glycerol tricarbamate is from 0.5:1 to 4.5:1, said condensate prepared in an aqueous medium by reacting at a pH 5.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

21. A thermosetting resin composition comprising a 0.5%-100.0% aqueous dispersion or solids, or combination thereof, made by reacting a mixture comprising meso-erythritol tetracarbamate and formaldehyde to form a condensate, in which the molar ratio of formaldehyde to meso-erythritol tetracarbamate is from 0.5:1 to 5.5:1, said condensate prepared in an aqueous medium by reacting at a pH 5.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

22. A thermosetting resin composition comprising a 0.5%-100.0% aqueous dispersion or solids, or combination thereof, made by reacting a mixture comprising D-xylitol pentacarbamate and formaldehyde to form a condensate, in which the molar ratio of formaldehyde to D-xylitol pentacarbamate is from 0.5:1 to 6.5:1, said condensate prepared in an aqueous medium by reacting at a pH 5.5-10.0, optionally followed by reacting at a pH 0.5-5.5, or reacting only at a pH 0.5-5.5, at a temperature of at least 30° C., and having a viscosity at a resins solids concentration of 60% in water at 25° C. of from 1.0 cP or higher, and optionally adding an alkaline material to the condensate to raise the pH to a value of 3.5-11.0.

23. A thermosetting resin composition comprising a 0.5%-100.0% aqueous dispersion or solids, or combination thereof, made by reacting a mixture comprising D-sorbitol hexacarbamate and formaldehyde to form a condensate, in which the molar ratio of formaldehyde to D-sorbitol hexacarbamate is from 0.5:1 to 7.5:1, said condensate prepared in an aqueous medium by reacting at a pH 5.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

24. A thermosetting resin composition comprising a 0.5%-100.0% aqueous dispersion or solids, or combination thereof, made by reacting a mixture comprising D-mannitol hexacarbamate and formaldehyde to form a condensate, in which the molar ratio of formaldehyde to D-mannitol hexacarbamate is from 0.5:1 to 7.5:1, said condensate prepared in an aqueous medium by reacting at a pH 5.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

25. A thermosetting resin composition comprising a 0.5%-100.0% aqueous dispersion or solids, or combination thereof, made by reacting a mixture comprising 1,1,1-trihydroxymethylethane tricarbamate and formaldehyde to form a condensate, in which the molar ratio of formaldehyde to 1,1,1-trihydroxymethylethane tricarbamate is from 0.5:1 to 4.5:1, said condensate prepared in an aqueous medium by reacting at a pH 0.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

26. A thermosetting resin composition comprising a 0.5%-100.0% aqueous dispersion or solids, or combination thereof, made by reacting a mixture comprising 1,1,1-trihydroxymethylpropane tricarbamate and formaldehyde to form a condensate, in which the molar ratio of formaldehyde to 1,1,1-trihydroxymethylpropane tricarbamate is from 0.5:1 to 4.5:1, said condensate prepared in an aqueous medium by reacting at a pH 5.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

27. A thermosetting resin composition comprising a 0.5%-100.0% aqueous dispersion or solids, or combination thereof, made by reacting a mixture comprising pentaerythritol tetracarbamate and formaldehyde to form a condensate, in which the molar ratio of formaldehyde to pentaerythritol tetracarbamate is from 0.5:1 to 6.5:1, said condensate prepared in an aqueous medium by reacting at a pH 0.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

28. A thermosetting resin composition comprising a 0.5%-100.0% aqueous dispersion or solids, or combination thereof, made by reacting of a mixture comprising ethylene glycol dicarbamate and formaldehyde to form a condensate, in which the molar ratio of formaldehyde to ethylene glycol dicarbamate is from 0.5:1 to 3.5:1, said condensate prepared in an aqueous medium by reacting at a pH 5.5-10.0, optionally followed by reacting at a pH 0.5-5.5, or reacting only at a pH 0.5-5.5, at a temperature of at least 30° C., and having a viscosity at a resin solids concentration of 60% in water at 25° C. of from 1.0 cP or higher, and optionally adding an alkaline material to the condensate to raise the pH to a value of 3.5-11.0.

29. A thermosetting resin composition comprising a 0.5%-100.0% aqueous dispersion or solids, or combination thereof, made by reacting of a mixture comprising ethylene dicarbamide and formaldehyde to form a condensate, in which the molar ratio of formaldehyde to ethylene dicarbamide is from 0.5:1 to 3.5:1, said condensate prepared in an aqueous medium by reacting at a pH 6.9-10.0, optionally followed by reacting at a pH 0.5-6.9 or reacting only at a pH 0.5-6.5, at a temperature of at least 30° C., and having a viscosity at a resin solids concentration of 60% in water at 25° C. of from 1.0 cP or higher, and optionally adding an alkaline material to the condensate to raise the pH to a value of 5.0-11.0.

30. A thermosetting resin composition comprising a 0.5%-100.0% aqueous dispersion or solids, or combination thereof, made by reacting of a mixture comprising urea, ethylene dicarbamide, and formaldehyde to form a condensate, in which the molar ratio of formaldehyde to combined urea and ethylene dicarbamide is from 0.5:1 to 3.0:1 and the molar ratio of urea to ethylene dicarbamide is from 0.05:1 to 1:1, said condensate prepared in an aqueous medium by reacting at a pH 6.9-10.0, optionally followed by reacting at a pH 0.5-6.9, or reacting only at a pH 0.5-6.9, at a temperature of at least 30° C., and having a viscosity at a resin solids concentration of 60% in water at 25° C. of from 1.0 cP or higher, and optionally adding an alkaline material to the condensate to raise the pH to a value of 5.0-11.0.

31. A thermosetting resin composition comprising a 0.5%-100.0% aqueous dispersion or solids, or combination thereof, made by reacting of a mixture comprising diethylene tricarbamide and formaldehyde to form a condensate, in which the molar ratio of formaldehyde to diethylene tricarbamide is from 0.5:1 to 6.0:1, said condensate prepared in an aqueous medium by reacting at a pH 6.9-10.0, optionally followed by reacting at a pH 0.5-6.9, or reacting only at a pH 0.5-6.9, at a temperature of at least 30° C., and having a viscosity at a resin solids concentration of 60% in water at 25° C. of from 1.0 cP or higher, and optionally adding an alkaline material to the condensate to raise the pH to a value of 5.0-11.0.

32. A thermosetting resin composition comprising a 0.5%-100.0% aqueous dispersion or solids, or combination thereof, made by reacting of a mixture comprising urea, diethylene tricarbamide, and formaldehyde to form a condensate, in which the molar ratio of formaldehyde to combined urea and diethylene tricarbamide is from 0.5:1 to 6.0:1 and the molar ratio of urea to diethylene tricarbamide is from 0.05:1 to 1:1, said condensate prepared in an aqueous medium by reacting at a pH 6.9-10.0, optionally followed by reacting at a pH 0.5-6.9, or reacting only at a pH 0.5-6.9, at a temperature of at least 30° C., and having a viscosity at a resin solids concentration of 60% in water at 25° C. of from 1.0 cP or higher, and optionally adding an alkaline material to the condensate to raise the pH to a value of 5.0-11.0.

33. A thermosetting resin composition made by mixing in any proportion two or more thermosetting resin compositions of claims 20-32.

34. A thermosetting resin composition made by mixing in any proportion at least one thermosetting resin composition of claims 20-32 with 0.05-5.0 moles of urea, urea-formaldehyde, melamine, melamine-formaldehyde, or melamine-urea-formaldehyde resin per mole of polycarbamide or polycarbamate in the resin.

35. A thermosetting resin composition comprising a cured product obtained by curing at least one thermosetting resin composition of claims 20-34 by adding an acid material in a 0.01%-10.0% level based on the resin solids level, or optionally by adding no acid material, and by heating at 80° C.-300° C. until the resin cures completely.

36. A wood composite product made by dispersing or spreading at least one thermosetting resin composition of claims 20-34 and by adding an acid material in a 0.01%-10.0% level based on the resin solids level, or optionally by adding no acid material, wherein the resin is cured by heating at 80° C.-300° C. until the resin cures completely.

37. The wood composite product of claim 36, wherein the product is comprised of wood particleboard consisting of any wood species, any particle size, and any density and thickness.

38. The wood composite product of claim 36, wherein the product is comprised of medium density fiber board consisting of any wood species, any fiber size, and any density and thickness.

39. The wood composite product of claim 36, wherein the product is comprised of hardwood or softwood plywood consisting of any wood species.
